(19) United States
(12) Patent Application Publication
(10) Pub. No.: US 2024/0168015 A1
(43) Pub. Date: May 23, 2024

(54) METHOD FOR THE DETECTION OF ORGAN DERIVED EXTRACELLULAR VESICLES

(71) Applicant: Hoffmann-La Roche Inc., Little Falls, NJ (US)

(72) Inventors: Priscila CAMILLO TEIXEIRA, Basel (CH); Axel DUCRET, Reinach (CH); Souphalone LUANGSAY, Basel (CH); Johanna Marie WALTHER, Basel (CH)

(73) Assignee: Hoffmann-La Roche Inc., Little Falls, NJ (US)

(21) Appl. No.: 18/424,571

(22) Filed: Jan. 26, 2024

Related U.S. Application Data
(63) Continuation of application No. PCT/EP2022/071015, filed on Jul. 27, 2022.

(30) Foreign Application Priority Data
Jul. 29, 2021 (EP) 21188404.4

(51) Int. Cl. G01N 33/569

(52) U.S. Cl. CPC G01N 33/56966; G01N 2333/705; G01N 2800/26

(57) ABSTRACT

The present invention provides a method for the detection of organ derived extracellular vesicles expressing PD-L1 in a biological sample

### CROSS REFERENCE TO RELATED APPLICATIONS

This application is a continuation of International Patent Application No. PCT/EP2022/071015, filed Jul. 27, 2022, which claims priority to European Patent Application number 21188404.4 filed Jul. 29, 2021, both of which are incorporated herein by reference in their entirety.

### FIELD OF INVENTION

The present invention relates to an assay for the detection of organ derived extracellular vesicles expressing PD-L1 (Programmed cell death 1 ligand 1) protein.

Over-expression of PD-L1 within the liver participates in local immune dysfunction and chronicity of HBV infection.

Having access to non-invasive biomarkers to follow the disease progression or a targeted therapy is an unmet medical need for patients. The exosomes can be used as a novel biomarker for patient monitoring and be a safer alternative to the need of a painful and stressful organ biopsy that requires several days to completely heal from the procedure.

Our goal is to explore methods for detection of liver derived exosomes in human serum in order to assess the biomarker modulation by liver-specific compounds

### DESCRIPTION OF THE INVENTION

The present invention provides a method for the detection of organ derived extracellular vesicles expressing PD-L1 in a biological sample comprising:
a) providing a biological sample,
b) contacting the biological sample with an antibody recognizing asialoglycoprotein receptor 1 (ASGPR1) protein,
c) contacting the biological sample with an antibody recognizing PD-L1 protein and
d) detection of extracellular vesicles expressing both ASGPR1 and PD-L1.

In an embodiment of the method, the biological sample is selected from blood, urine, saliva and CSF.

In an embodiment of the method, the organ derived extracellular vesicles are exosomes.

In an embodiment of the method, the extracellular vesicles are enriched in the biological sample.

In an embodiment of the method, the detection of extracellular vesicles expressing both ASGPR1 and PD-L1 is done by ELISA.

In an embodiment of the method, the ELISA uses the biotin streptavidin system.

In an embodiment of the method, the ASGPR1 recognizing antibody is MAB43941 and the PD-L1 antibody is NBP1-76769.

In an embodiment of the method, the organ derived extracellular vesicles are liver derived extracellular vesicles.

In an embodiment of the method, the biological sample is a biological sample of a human patient.

In an embodiment of the method, both ASGPR1 and PD-L1 proteins are human proteins.

Extracellular vesicles (EV) are a heterogeneous group of cell-derived membranous structures comprising exosomes and microvesicles, which originate from the endosomal system or which are shed from the plasma membrane, respectively. EV can easily be isolated from biological fluids such as plasma, serum, urine or CSF.

The present invention provides an assay for quantifying cell-surface analytes carried by a defined population of extracellular vesicles (EV) using an enzyme linked imunosorbent assay (ELISA). The assay takes advantage that EV carry proteins that are characteristic for their lineage and their possible interaction partners on their cell surface membrane (van Niel, G., D'Angelo, G. & Raposo, G. Shedding light on the cell biology of extracellular vesicles. Nat Rev Mol Cell Biol 19, 213-228 (2018). https://doi.org/10.1038/nrm.2017.125). Thus, assay specificity is provided by capturing first the EV population of interest before quantifying a specific analyte on them.

The general assay setup is shown in FIG. 1. In a first step, a capture antibody specific for a protein carried out by the selected EV population (in this example: the membrane-bound ASGPR1, which is specific for a liver origin) is immobilized in a microtiter plate (for example, by using a biotinylated antibody on a streptavidin-coated microtiter plate). In a next step, an EV-containing sample (for example, an EV preparation that has been enriched from serum using a commercially-available precipitation kit) is incubated in the well and the EV population carrying the targeted protein remain in the well. This is followed by a third step, in which the immobilized EVs are targeted by an antibody specific for the analyte of interest (in this example, an anti-PD-L1 antibody, if the PD-L1 density on liver-specific EVs is measured). Quantification of the analyte is carried out using a commonly used luminescent, fluorescent, or colorometric reaction using for example a peroxidase-coupled secondary antibody targeted for the analyte-specific antibody.

Despite their relative abundance, EVs analysis benefit from being enriched from bio fluids proteins, cell debris and other types of vesicles, and also to remove the potential non-membrane bound version of the analyte of interest. There are a number of methods available for this purpose. In this example, we use a simple polymer-based precipitation followed by a protein depletion column to obtain a highly-enriched EV fraction. Other methods of purification, such as size-exclusion chromatography-based or ultracentrifugation-based, can be used as well.

### SHORT DESCRIPTION OF THE FIGURES

FIG. 1: General setup for a population-specific EV ELISA assay. In this example, the assay is setup to quantify the PD-L1 density on liver-derived EVs (as characterized by the presence of the ASGPR1 cell surface receptor).

FIG. 2: Isolation of EV from serum samples using the ExoQuick® ULTRA EV Isolation Kit (SBI).

FIG. 3 show a Generic population—specific EV assay setup

FIG. 4 shows the analysis of CD63 positive EVs. In this example, we demonstrate the size tracking of EV particles enriched using the ExoQuick® ULTRA EV Isolation Kit (SBI). The subset of the particles labelled with a fluorescent antibody against CD63, a hallmark of EV particles, shows the expected particle size distribution of 80-120 nm.

FIG. 5 shows the robustness and the linearity of the ASGPR1/PD-L1 hybrid exosome ELISA using two separate calibrators. A serial dilution of EV particles (in arbitrary unit, A.U.) were measured using the ASGPR1/PD-L1 hybrid exosome ELISA. Results are reported in mean optical density (O.D.).

FIGS. 6A and 6B show that a specific signal is detected only in presence of the capture antibody. FIG. 6A: results of ASGPR1 total ELISA assay. No signal was detected in absence of capture antibody (ON_1 μg/ml: overnight incubation with 1 μg/ml capture antibody; ON_w/o: overnight incubation in absence of capture antibody). FIG. 6B: results of ASGPR1/PDL1 hybrid ELISA. No signal was detected in absence of a specific capture antibody (ON_1 μg/ml: overnight incubation with 1 μg/ml capture antibody; ON_1 μg/ml isotype control: overnight incubation with antibody isotype control). Results for 3 different donors are shown.

FIG. 7 shows the density of PD-L1 on ASGPR1-containing EVs in donor samples measured using the ASGPR1/PDL1 hybrid ELISA assay.

### EXAMPLES

Isolation of EV Particles

The following EV preparation protocol has been used to measure organ-specific or functionally-related extracellular vesicles (EV) particles using the generic enzyme linkedELISA assay. Some modifications to the original kit were added by Microcoat to increase reproducibility of the EV isolation protocol and to reduce protein interference.

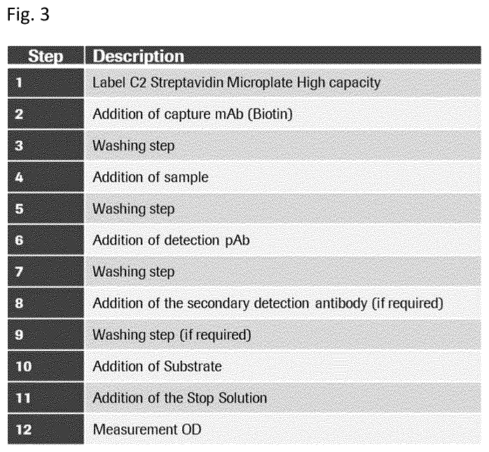

EVs isolation from human serum is based on the ExoQuick® ULTRA EV Isolation Kit for Serum and Plasma (EQULTRA-20A-1, SBI, Palo Alto, CA, USA). This kit is an ultracentrifugation-free method of isolating EVs from biofluids based on precipitating EVs with a proprietary polymer and a subsequent column-purification step. EVs isolated at Microcoat was performed according to the manufacturer's protocol with slight modifications as shown in FIG. 2. In brief, serum samples are first centrifuged to remove cell debris. The resulting supernatant is then incubated with the kit-specific proprietary polymer (“ExoQuick” buffer) and the EVs pelleted by centrifugation. The pellet is re-suspended in the kit-specific Buffer B and further diluted with the kit-specific Buffer A. Here, modifications were made internally compared to the manufacturers protocol concerning the buffer volumes (see FIG. 2). The re-suspended EVs (column input) are then further purified (protein-depleted) by equally distributing their volume on three (instead of one originally intended) kit-specific Purification spin-columns. The flow-through of the three columns is finally pooled and further assayed.

Table 1 shows the antibody used in the examples of the present invention:

| | | | Ab capture | Ab detection | |
|---|---|---|---|---|---|
| Assay | Capture | Analyte | anti-ASGPR1 | anti-PD-L1 | Secondary detection Ab |
| Liver- | ASGPR1 | PD-L1 | MAB43941- | NBP1-76769 | Alpaca anti-rabbit HRP Ab, |
| specific | | | 100 (R&D | (Novus | 611-035-215, Jackson |
| EV PD-L1 | | | Systems) | Biologicals) | ImmunoResearch |

Assay Results:

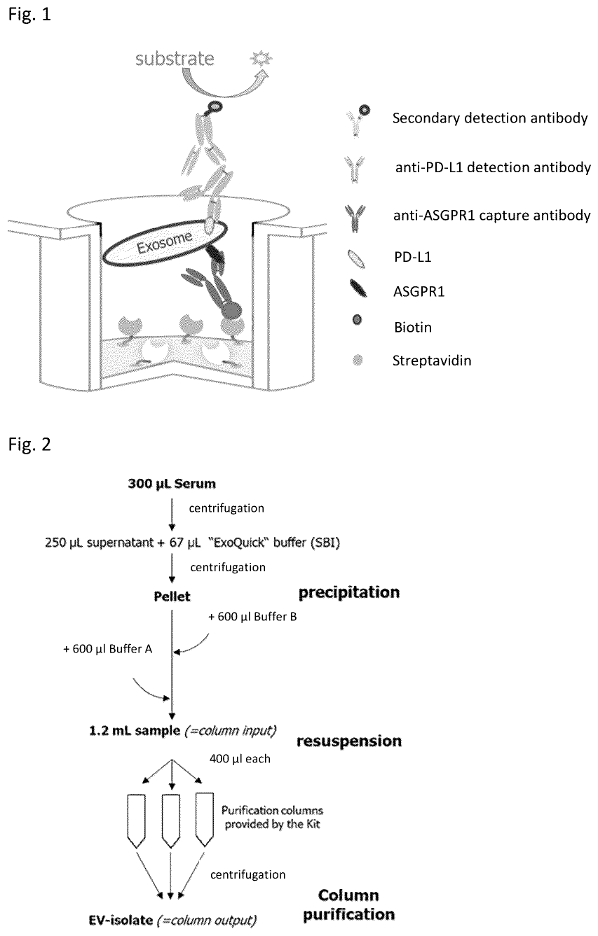

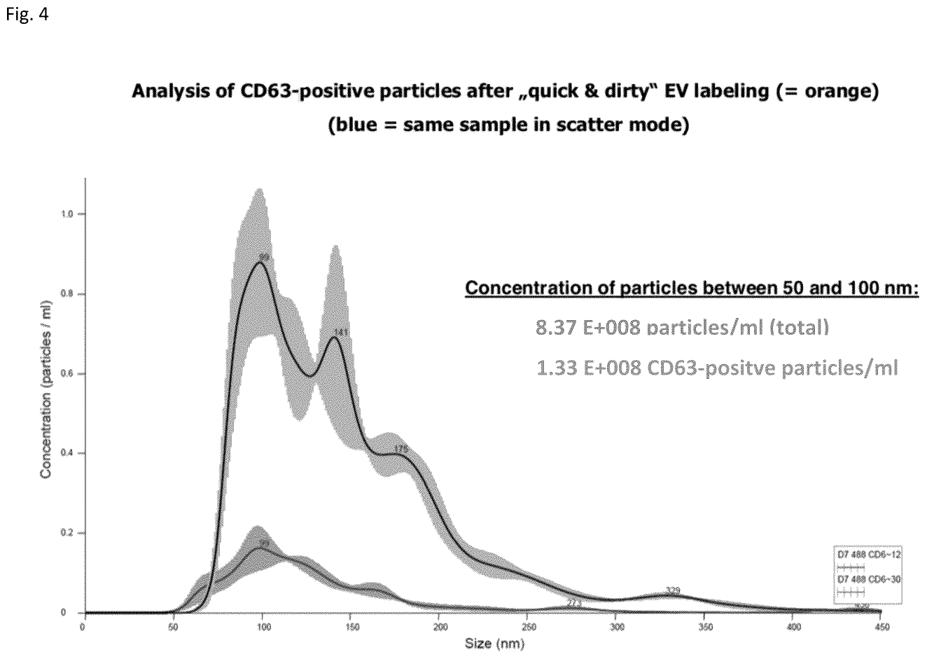

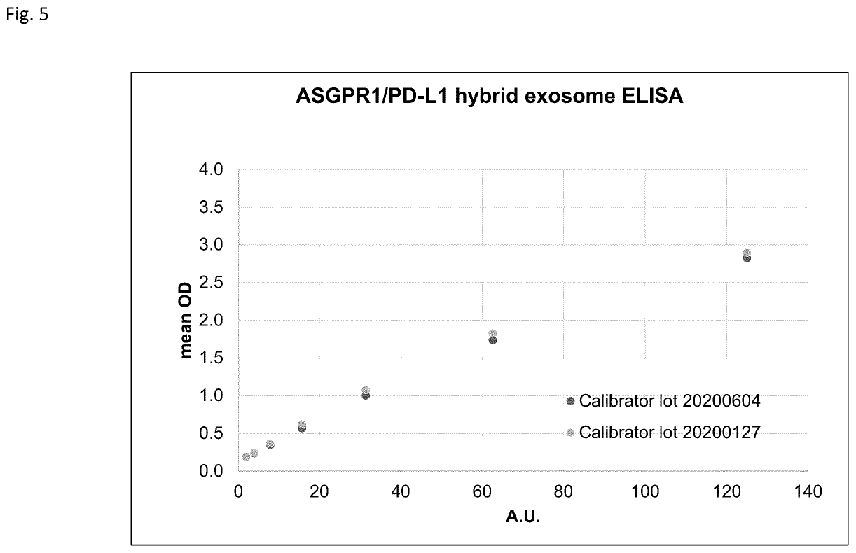

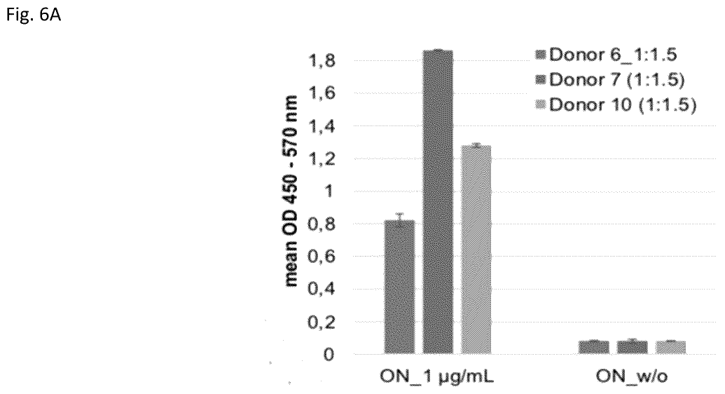

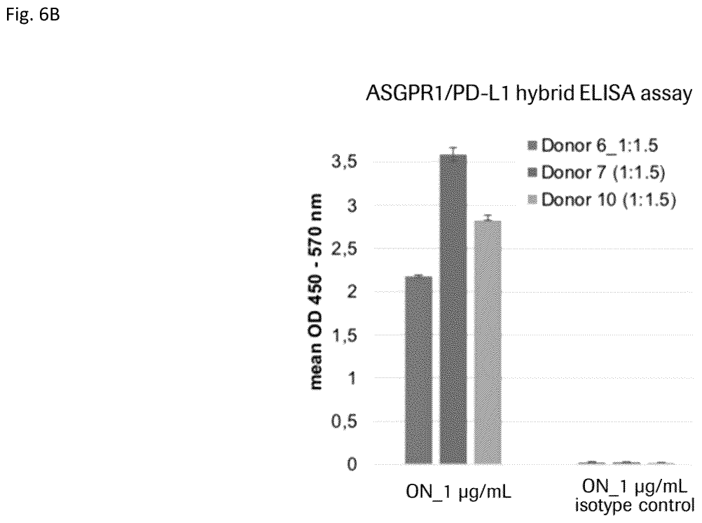

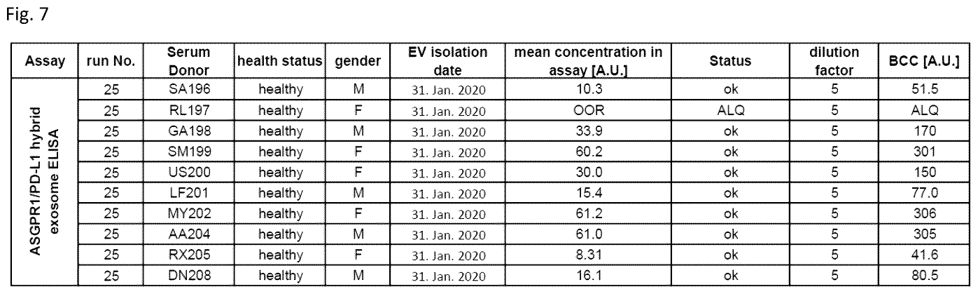

After Exoquick® ULTRA EV enrichment (FIG. 4), eluates are diluted with assay buffer to the recommended Minimal Required Dilution and measured using the appropriate hybrid ELISA to report on the selected analyte. For example, to report back on PD-L1 density on liver-expressed EV, such particles need to be first captured by the ASGPR1-coated streptavidin plate prior to detection using a PD-L1 specific antibody (FIG. 1, FIG. 3). As there is no commercially available standard available, such hybrid ELISA assays need to be calibrated using purpose-specific standard to determine the linearity and the robustness of the assay (FIG. 5). Typically, calibrators are a custom-made mixture of individual EV preparations to offer a harmonized standard offering a large dynamic range in all assays that are considered in a study. Specificity of the assay was demonstrated in showing that signals were only obtained in the presence of all reagents required for capture and detection of the EV particles. In this example (FIG. 6), the density of PD-L1 on liver-expressed EV is only obtained in the presence of a functional ASGPR1-capture antibody. FIG. 7 demonstrates the use of such an assay in the measurement of serum samples in a trial. Serum was collected and EV were enriched using the Exoquick® ULTRA EV kit. Samples were dilution in the recommended Minimal Required Dilution (here, a 5-fold dilution) and the density of PD-L1 on liver-expressed EV was measured using the PD-L1-ASGPR1 hybrid ELISA assay. In 9 out of 10 cases, in this example, a back-calculated concentration could be reported against the performance of a calibrator that was analyzed in the same analytical procedure. One sample (from donor RL197, FIG. 7) was measured out of range in the assay in this first measurement. The procedure is then to dilute the EV preparations of such donors further and to repeat measurement, until a valid concentration (in absolute unit) is obtained.

1. A method for the detection of organ derived extracellular vesicles expressing PD-L1 in a biological sample comprising:
a. providing a biological sample,
b. contacting the biological sample with an antibody recognizing asialoglycoprotein receptor 1 (ASGPR1) protein,
c. contacting the biological sample with an antibody recognizing PD-L1 protein and
d. detection of extracellular vesicles expressing both ASGPR1 and PD-L1.

2. The method of claim 1, wherein biological sample is selected from blood, urine, saliva and CSF.

3. The method of claim 1, wherein the organ derived extracellular vesicles are exosomes.

4. The method of claim 1, wherein the extracellular vesicles are enriched in the biological sample.

5. The method of claim 1, wherein the detection of extracellular vesicles expressing both ASGPR1 and PD-L1 is done by ELISA.

6. The method of claim 5, wherein the ELISA uses the biotin streptavidin system.

7. The method of claim 1, wherein the ASGPR1 recognizing antibody is MAB43941 and the PD-L1 antibody is NBP1-76769.

8. The method of claim 1, wherein the organ derived extracellular vesicles are liver derived extracellular vesicles.

9. The method of claim 1, wherein the biological sample is a biological sample of a human patient.
